(19) United States
(12) Patent Application Publication
(10) Pub. No.: US 2006/0106362 A1
(43) Pub. Date: May 18, 2006

(54) Administration of insulin by jet injection

(76) Inventors: Franklin Pass, St. Paul, MN (US); Mario Velussi, Aurisina (IT)

Correspondence Address: WINSTON & STRAWN LLP, 1700 K STREET, N.W., WASHINGTON, DC, 20006, US

(21) Appl. No.: 11/117,317

(22) Filed: Apr. 29, 2005

Related U.S. Application Data
(63) Continuation of application No. PCT/US03/04062, filed on Feb. 12, 2003.
(60) Provisional application No. 60/422,850, filed on Nov. 1, 2002.

(51) Int. Cl. A61M 31/00

(52) U.S. Cl. 604/504; 604/68

(57) ABSTRACT

The invention relates to a method for minimizing mean blood glucose levels in an insulin dependent patient by administering insulin to the patient in a sufficiently fast manner to provide a difference of 50% or less between high and low blood glucose levels. Advantageously, the insulin is administered to the patient by jet injection and the high and low blood glucose levels differ by an amount that is less than that which would be obtained after injection of insulin by a conventional needle syringe. The invention also relates to a method for reducing mean blood glucose levels in an insulin dependent patient that is receiving insulin through a conventional syringe and needle arrangement. This method provides for administration of the insulin to the patient by jet injection rather than by the syringe by substituting a jet injector for the syringe.

### CROSS-REFERENCE TO RELATED APPLICATIONS

This application is a continuation of PCT Application No. PCT/US03/04062 filed Feb. 12, 2003, which claims priority to U.S. Provisional Patent Application No. 60/422,850 filed Nov. 1, 2002. The content of both applications is hereby incorporated herein by reference thereto.

### FIELD OF INVENTION

The invention relates to improved methods of managing blood glucose levels by needle-free insulin injection. More particularly, the invention is related to a method of administering insulin using a jet injection device, as well as a method of improving glycemic control in individuals in order to obtain enhanced management of blood glucose levels.

### BACKGROUND OF THE INVENTION

Diabetes generally refers to the group of diseases in which the body does not produce or properly use insulin, a hormone needed to convert sugar, starches, and other food into energy. Well over 16 million Americans alone are believed to have diabetes, and thus the prevalence of diabetes in the population needs not be further emphasized.

Diabetes results in elevation of the blood glucose level because of relative or absolute deficiency in the pancreatic hormone insulin, which is secreted into the blood when food is ingested and primarily directs absorbed nutrients into body stores. Of the various metabolic effects of diabetes, chronic elevation of the blood glucose level is the most prominent, and is associated with progressive damage to blood vessels. Higher mean glucose levels are associated with increased incidence of complications such as heart attack, stroke, blindness, peripheral nerve dysfunction, kidney failure, impotence, and skin disease. The goal of therapy is to reduce the mean glucose level. In doing so, however, the risk of hypoglycemic events and resulting central nervous system (CNS) complications may be increased.

In general, there are four primary types of diabetes, of which types 1 and 2 account for about 99% of the cases. In type 1 diabetes, the pancreas no longer produces insulin because the beta cells have been destroyed. Insulin shots are thus required so that glucose may be used from food. In type 2 diabetes, the body produces insulin, but does not respond well to it. Type 2 diabetes is typically treated with diabetes pills or insulin shots which assist the body in using glucose for energy. Insulin, however, cannot be administered as a pill, because it would be broken down during digestion similar to the protein in food. Thus, insulin must be injected.

A diverse range of insulins are administered for treatment of diabetes. Generally, four types of insulins are available, and are characterized based on how quickly the insulin reaches the blood and starts working (known as the “onset”), when the insulin works the hardest (known as the “peak time”), and how long the insulin lasts in the body (known as the “duration”). Each type of insulin produces a characteristic glucose profile in response to the combined effects of onset, peak time, and duration. The first type of insulin, rapid-acting insulin (Lispro), has an onset within 15 minutes following injection, has a peak time at about 30 to about 90 minutes later, and has a duration of as long as about 5 hours. The second type of insulin, short-acting (regular) insulin, has an onset within 30 minutes after injection, has a peak time at about 2 to about 4 hours later, and has a duration of about 4 to about 8 hours. A third type of insulin includes intermediate-acting (NPH and lente) insulins which have an onset with about 1.5 to about 3 hours after injection, have a peak time at about 4 to about 12 hours later, and have a duration of up to about 24 hours. Finally, the fourth type of insulin, long-acting (ultralente, Lantus/insulin glargine) insulin, has an onset within about 2.5 to about 8 hours after injection, has no peak time or a very small peak time at about 7 to about 15 hours after injection, and has a duration of up to about 24 hours or longer. The aforementioned data is highly variable, however, based on an individual's characteristics. Several of the insulins are sometimes mixed together for simultaneous injection.

Insulins are provided dissolved in liquids at different strengths. Most people, for example, use U-100 insulin, which has 100 units of insulin per milliliter (mL) of fluid. Initially, type 1 diabetics typically require two injections of insulin per day, and eventually may require three or four injections per day. Those individuals with type 2 diabetes, however, may only need a single injection per day, usually at night. Diabetes pills may, however, become ineffective for some people, resulting in the need for two to four injections of insulin per day. In general, the optimum way to treat type 1 patients and later-stage type 2 patients is to administer regular insulin prior to each meal and give a dose of intermediate acting insulin at bedtime. Optimization of treatment regimen though, is often at the discretion of doctor and patient.

Insulin is conventionally delivered through the skin using a needle on a catheter that can be connected to a pump, on a syringe, on a pen to penetrate the skin prior to injection. Individuals often find syringe use to be uncomfortable, difficult, or even painful. Insulin pens have been developed which permit insulin to be administered by dialing a desired dose on a pen-shaped device, which includes a needle through which the insulin is subsequently injected.

A small segment of the insulin injection market, i.e., about 1%, utilizes jet injectors to administer insulin. The people who receive insulin injections by jet injectors are either afraid of needles or are interested in new technology. The relative amount of jet injector administration users has not significantly increased over the years, possibly because most diabetics have become used to the syringe needle injection form of administration or because they see no advantage for utilizing jet injectors. The present invention now overcomes a number of problems associated with the use of conventional syringes and provides enhanced performance when insulin is administered utilizing jet injections, and it is believed that these benefits will lead to much greater use of jet injector devices for the administration of insulin.

### SUMMARY OF THE INVENTION

The invention relates to a method for minimizing mean blood glucose levels in an insulin dependent patient by administering insulin to the patient by jet injection to provide high and low blood glucose levels that differ by an amount that is less than that which would be obtained after injection of insulin by needle injection, such as by a conventional needle syringe. Advantageously, the insulin is administered to the patient in a sufficiently fast manner to provide a difference of 50% or less between high and low blood glucose levels. When U-100 insulin is used, preferably about 2 to 50 units, which is about 0.02 mL to 0.5 mL of insulin, is administered to the patient. The injector preferably is configured such that 0.05 mL of saline takes less than about 0.5 seconds to be expelled from the syringe with a 0.0065 in jet nozzle orifice. Other orifice sizes can be used. The speed for ejecting U-100 insulin into air is preferably similar. Preferably, the syringe is configured to eject this amount of fluid in at most about 0.3 seconds, more preferably in at most about 0.25 seconds, and most preferably in at most about 0.2 seconds.

In a preferred embodiment, the difference between high and low blood glucose levels is about 25% or less. Also, the high blood glucose level is less than about 200 mg/dL.

Preferably, the blood glucose levels are reduced to minimum differences between the high and low levels over a period of about 1 week. A preferred device for administering the insulin to the patient is a jet injector that is easy to use by an unassisted patient.

In another embodiment, the invention relates to a method of treatment of a medical condition caused by elevated blood glucose levels in an insulin dependent patient which comprises minimizing mean blood glucose levels in the patient by the method described. In yet another embodiment, the invention relates to a method for reducing an insulin dependent patient's HbA1c value which comprises minimizing mean blood glucose levels in the patient by the method described previously, thus reducing the patient's HbA1c value.

The invention also relates to a method for reducing mean blood glucose levels in an insulin dependent patient that is receiving insulin through a conventional syringe and needle arrangement. This method provides for administration of the insulin to the patient by jet injection rather than by the syringe, which improves the patient's glucose level. This can be done by substituting a jet injector for the syringe. The advantages and features of the previously described embodiments can be used in this embodiment as well.

Another embodiment of the invention relates to a method for reducing mean blood glucose levels in an insulin dependent patient that is receiving insulin through needle injection. This method comprises administering the insulin to the patient by jet injection rather than by the needle injection or substituting a jet injector for a needle injection assembly for administration of the insulin so that HbA1c levels can be reduced by at least 5% to about 8% over a period of 6 months. Furthermore, HbA1c levels are reduced by at least 10% to as much as 14% over a period of one year.

The invention also relates to a method for reducing nocturnal mean blood glucose levels in an insulin dependent patient by administering insulin to the patient by jet injection prior to bedtime to reduce mean blood glucose through the night and to produce a less-pronounced blood glucose nadir in the early morning hours, thus reducing the risk of nighttime hypoglycemia. In this method, the difference between high and low blood glucose levels during the night is about 25% of the high level or less, and the high blood glucose level is less than about 200 mg/dL. Also, the mean blood glucose levels do not exceed the level at the time of injection for at least 5 to about 8 hours.

In these embodiments, the insulin is preferably administered to the patient from a jet injector that comprises a jet nozzle configured for firing the insulin in a fluid jet configured and with sufficient velocity to penetrate tissue of the patient to an injection site; an insulin chamber associated with the nozzle for containing the insulin and feeding the insulin to the nozzle for injection; a firing mechanism comprising an energy source associated with the insulin chamber for forcing the insulin through the nozzle at said velocity; and a trigger movable by a user and associated with the firing mechanism for activating the energy source for the forcing of the insulin through the nozzle upon movement of the trigger by the user to a firing position.

The invention provides an effective way of administering insulin in a manner that is easy for a patient user to employ without needing a high level of skill. The invention can improve glycemic control in individuals, even those who are already well-controlled individuals, in order to obtain enhanced management of blood glucose levels.

### BRIEF DESCRIPTION OF THE DRAWINGS

The invention will be better understood in relation to the attached drawings illustrating preferred embodiments, wherein:

FIG. 1 is a cross-sectional lateral view of a preferred embodiment of an injector used in accordance with the invention;

FIG. 2 is a cutaway lateral view of an adapter connected to a vial of insulin and to the nozzle of the preferred injector;

FIG. 3 is a perspective view of the adapter;

FIG. 4 is a perspective view of the nozzle;

FIG. 5 is a lateral cross-sectional view of a rear portion of the injector showing the trigger and safety mechanisms;

FIGS. 6-8 are a perspective, lateral, and rear end view of the injector, respectively;

FIG. 9 shows a graphical comparison of experimental test results of blood glucose levels in mg/dL after administration of insulin as a fraction of time of day using a pen device equipped with a needle and an Antares Pharma Vision jet injection device for administration of insulin over a three day period;

FIG. 10 shows a graphical representation of the difference in blood glucose levels obtained using the Vision jet injector and pen devices in the experimental study presented in FIG. 9, with blood glucose level in mg/dL plotted as a function of time of day;

FIG. 11 shows a graphical representation of the mean blood glucose levels obtained using the Vision jet injector and pen devices in the experimental study presented in FIG. 9, with blood glucose level in mg/dL plotted as a function of the device;

FIG. 12 is a graphical comparison of the difference in blood glucose levels obtained using a pen device with a needle and a jet injector over a one year period;

FIG. 13 is a graphical representation of HbA1c levels obtained using a jet injector device over the one year period mentioned in FIG. 12; and

FIG. 14 is a graph that compares nighttime blood glucose values in patients after NPN injection by pen and jet injection devices

### DETAILED DESCRIPTION OF THE PREFERRED EMBODIMENTS

As used herein, “insulin-dependent” means that the patient is receiving treatment for elevated blood glucose by oral or intramuscular administration of insulin or other hypoglycemic agents. “Well-managed patients” are those who faithfully follow instructions from their doctors and pharmacists for the daily administration of insulin or other hypoglycemic agents. Such patients typically have HbA1c values of 7 or less.

Needle-free injection devices generally contemplated for use with the present invention (known in the art as “jet injectors”) are disclosed, for example, in U.S. Pat. No. 5,599,302, the content of which is expressly incorporated herein by reference thereto. One exemplary device for use with the present invention is the Antares Pharma Vision Needle-Free Insulin Injection System, manufactured by Antares Pharma of Minneapolis, Minn. This precision, needle-free drug delivery system uses pressure to create a micro-thin stream of insulin that penetrates the skin and is deposited into the subcutaneous (fatty) tissue in a fraction of a second. The device permits dialing of dosages, and easy injection without the use of a needle.

As insulin is often injected by a patient him or herself, the preferred method employs an injector that facilitates the proper insulin administration by the patient without the experience that a health provider would normally have. Although the patient is the typical user envisioned, other users are envisioned as well.

The preferred injector for administering the insulin has a jet nozzle configured for firing the insulin in a fluid jet in a configuration and with sufficient velocity to penetrate tissue of the patient to an injection site. A chamber is associated with the nozzle for containing the insulin and feeding the insulin to the nozzle for injection. This chamber is referred to herein as an insulin chamber as in the preferred method insulin is contained. A firing mechanism comprising an energy source is associated with the insulin chamber for forcing the insulin through the nozzle at said velocity. Although the energy source of the preferred embodiment is a coil spring, other suitable energy sources including other springs can be used. A trigger of the injector is movable by the patient and associated with the firing mechanism for activating the energy source for the forcing of the insulin through the nozzle upon movement of the trigger by the patient to a firing position.

The injector also has a safety mechanism with a blocking member that has a blocking position in which the blocking member prevents movement of the trigger to the firing position. A user-manipulable member of the safety mechanism is movable by the user from a safety position, allowing the blocking member to be positioned in the safety position, to a release position. In the release position, the manipulable portion is associated with the blocking member to move the blocking member to enable movement of the trigger to the firing position. The movement of the trigger with respect to the firing position preferably moves the manipulable member to the safety position, and preferably the movement of the trigger to the firing position moves the manipulable member to the safety position.

The manipulable portion is moved in a first direction from the release position to the safety position, and the trigger is preferably moved in substantially the first direction towards the firing position to activate the energy source. The manipulable member is preferably moved to cause resilient movement of the blocking member from the blocking position. The blocking member itself is naturally resiliently spring-biased toward the blocking position.

A latch member is preferably interposed with the firing mechanism for preventing the activation of the energy source, and the trigger is moved to the firing position to release the latch member from the firing mechanism to enable the activation of the energy source. The preferred location of the safety member and the trigger is near an axial end of the injector opposite from the nozzle, with the safety member and trigger mounted on a portion of the injector that is rotatable with respect to the nozzle to load the insulin into the chamber.

A housing of the injector used in the preferred method is associated with the trigger and has an axial cross-section that is generally triangular to facilitate the patient's grip during operation of the injector. The axial cross-section of this embodiment has rounded sides for comfortably holding in the patient's or other user's hand. This axial cross-section also comprises a lobe protruding at each apex of the cross-section configured and dimensioned for fitting adjacent the inside of the patient's knuckles during the injection. A preferred housing associated with the trigger has an elastomeric surface disposed and configured for facilitating the users' grip and control of the injector during the injection.

To facilitate the loading of the insulin into the injector, the complexity of motions is minimized to connect an adapter to the injector to load the insulin. In a preferred method, the adapter is attached to the needless injector to place an insulin passage of the adapter in fluid communication with the jet nozzle. The attaching preferably includes pushing the adapter against the nozzle without substantial relative rotation therebetween to engage the adapter and nozzle with respect to each other to keep the insulin passage in fluid association with the nozzle. The insulin chamber of the injector is then filled through the adapter and nozzle.

The preferred adapter used has a first engagement portion, and the injector has a second engagement portion. One of the engagement portions is resiliently displaced by the other engagement member when the adapter is moved against the nozzle. This causes the one engagement member to move to an engagement position in which the first and second engagement members are engaged with each other to keep the insulin passage in fluid communication with the nozzle. Preferably, the nozzle has an axis and attaching the adapter involves pushing the adapter against the nozzle so any relative rotation therebetween is at an angle of at most about 15° tangential to the axis. To achieve this, the at least one of the injector and adapter can have a slot, with the other having a protrusion that is received in the slot during the attachment. The slot is preferably substantially straight and configured for guiding and retaining the protrusion when the adapter is attached with the nozzle. In a preferred embodiment, the nozzle is attachable to a power pack portion of the injector by relative rotation therebetween

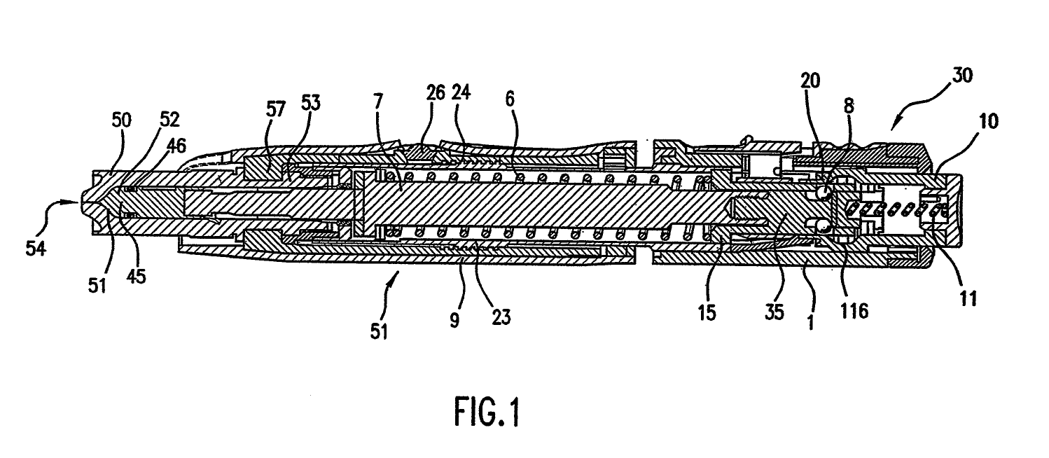

As noted above, the most preferred jet injector for the invention is the Antares Pharma Vision Needle Free Injection Device although other jet injectors with similar features can be used if desired. Referring to FIG. 1, a preferred embodiment of an inventive needleless jet injector has an actuating mechanism 30, preferably at a proximal side of the injector. This jet injector is the Antares Pharma Vision Device. The actuating mechanism 30 preferably includes a proximal injector housing 1 attached to a sleeve 23, which can by rotated relative to distal injector housing 9.

The actuating mechanism 30 has a prefiring condition, which is shown in FIG. 1. In this position, a trigger wall 20 of trigger button 10 retains a latch member, such as balls 8, interposed between a housing latch 15, which is preferably fixed with respect to the sleeve 23, and firing ram 7. In the prefiring condition, ram 7 retains firing spring 6 in compression.

At the forward, distal end of the injector is a nozzle assembly 50 that includes an insulin chamber 52, configured for containing insulin to be injected. A plunger 45, including seal 46 that seals against the wall of the insulin chamber 52, is received in the chamber 52 and is shown in a preloading position. The nozzle assembly 50 includes a jet nozzle orifice 54 configured for firing the insulin from the chamber 52 in a fluid jet sufficient to penetrate tissue of the patient to an injection site. Preferably, a skin contacting protrusion, such as ring 55, extends around the orifice 54 to apply pressure on a predetermined area around the skin to improve insulin delivery to the injection site.

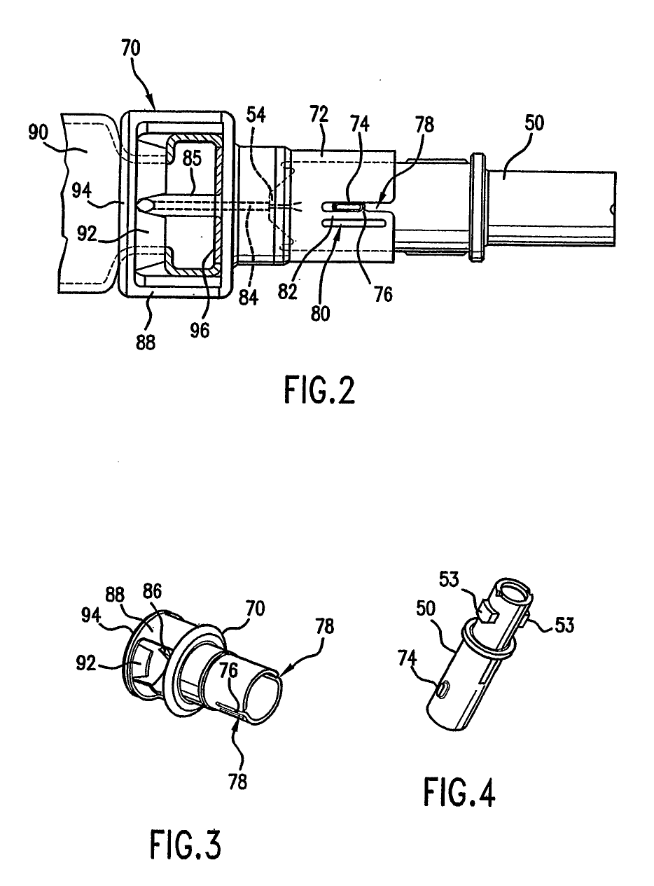

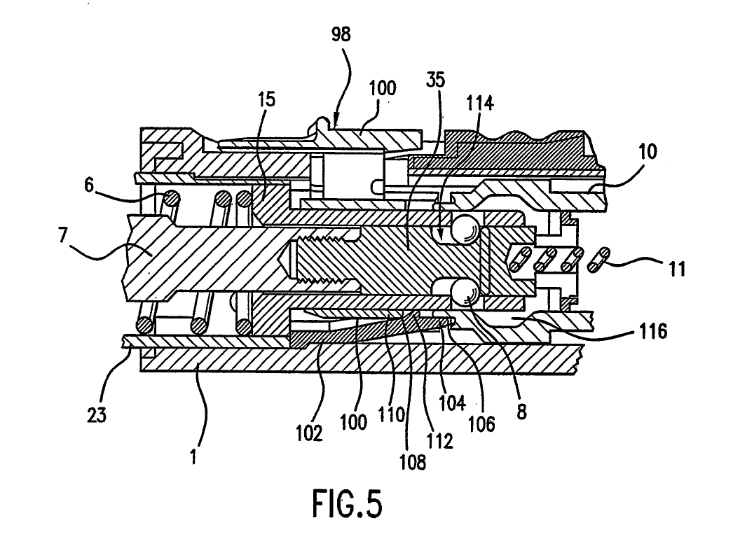

To fill the injector, an adapter 70 is attached to the distal end of the injector, preferably to nozzle 50, as shown in FIG. 2. Referring to FIGS. 2-4, the adapter 70 has a nozzle attachment sleeve 72 that is configured to receive nozzle 50 and to form a seal therewith. The attachment sleeve 72 and the nozzle 50 have engagement members, which preferably include a post 74 or other protrusion, preferably extending from the nozzle 50, and a resiliently biased catch 76. The catch 76 is disposed adjacent to and facing slot 78 formed in the sleeve 72. The slot has a width preferably corresponding to the tangential width of the post 74 to guide the post 74 as it is inserted into the slot 78 and to hold the post 74 in engagement against the catch 76. The catch 76 has front and rear ramps to enable the post 74 to be pushed in or out of engagement therewith, and extends from a resilient portion 82 of unitary construction with the sleeve 72, opposite an opening 80 to provide resilience and spring characteristics to the resilient portion 82. The resilient portion is preferably attached to the remainder of the sleeve 72 at two axial ends on opposite sides of the catch 76.

To attach the adapter 70 to the nozzle 50, the patient or other user pushes the adapter 70 against the nozzle, preferably without substantial relative rotation therebetween. This facilitates the engagement of the adapter 70 and nozzle 50 by the patient, preferably without requiring complex motions in various directions or substantial twisting motions. Thus, the slot 78 is preferably substantially straight, and any relative rotation between the nozzle 50 and adapter 70 is preferably at a pitch angle of at most about 15° tangential to the axis and more preferably at most about 10°. In addition, the snap fit of the engagement portions provides the patient or user with an indication that the adapter is properly attached to load insulin into the insulin chamber 52.

Preferably, the nozzle 50 is attached by a bayonet fitting to the power pack 51 of the injector, which includes the housings 1,9, the energy source, and the actuating mechanism 30. The bayonet fitting includes lugs 53 on the nozzle 50 and walls 57 within the distal housing 9. To attach the bayonet fitting, the nozzle 50 is pushed into the distal housing 9, and then rotated to engage the lugs 53 behind a wall 57 of the power pack 51. Preferably, the motion of the adapter 70 relative to the nozzle 50 to attach the adapter 70 is in a different direction than the motion to attach the nozzle 50 to the power pack 51, and preferably only one of these attachment motions requires any substantial twisting. This reduces potential confusion of the user about whether the adapter 70 and the nozzle 50 are attached properly.

When the adapter 70 is attached to the injector, an insulin passage 84 of the adapter 70 is in fluid communication with the jet nozzle orifice 54. The insulin passage includes a needle bore of needle 86, which extends into an ampule attachment portion 88 of the adapter 70. The ampule attachment portion 86 is configured for association with an ampule 90 to extract the contents of the ampule 90, which is preferably insulin, for delivery to the chamber 52. Tabs 92 of the-ampule attachment portion 90 extend inwardly from an outer support 94 of the ampule attachment portion 86 and are resilient to engage en enlarged end of the ampule 90. When the ampule 90 is attached, the needle 86 pierces an end of the ampule 90, such as a rubber seal 96, and allows the transfer of the contents of the ampule 90 to the injector.

With the adapter 70 attached, the sleeve portion 23 is rotated with respect to the distal housing 9 about threads 24 to draw the plunger 45 distally with respect to the nozzle orifice 54, drawing medication into the ampule chamber 50. To purge any air that may be trapped in the chamber 52, the injector is held upright with the nozzle 50 facing up, and the sleeve 23 is turned slightly in the opposite direction. During filling, the desired dosage of the medication is withdrawn into the chamber 52 can be measured by reading a number printed on the sleeve 23 through a window 26.

Referring to FIG. 5, once the insulin is loaded into the chamber 52, a safety mechanism 98 keeps the injector from firing unintentionally. The safety mechanism 98 of the preferred embodiment includes a slider 100 that is manipulable by user. The slider 100 is disposed in the proximal portion of the injector and mounted to the proximal housing 1 at a distance from the portion of the trigger button 10 that is pushed to fire the injector selected, so that the slider 100 and the trigger button 10 can be operated by the same hand or finger, preferably while the injector is grasped by the patient in a manner that will enable positioning and firing of the injector into the injection site.

A blocking member 102 is shown disposed in a blocking position in which it prevents movement of a portion of the trigger, such as the trigger button 10, from moving to a firing position to fire the injector. The preferred blocking member 102 comprises a resilient plate that is biased inwardly behind a portion of the sleeve 100 and which is mounted to proximal housing 1. A blocking portion 104 of the blocking member 102 preferably abuts and is biased against the trigger button 10, and is stably receivable within recess 106 of the trigger button 10. When the slider I 00 is slid rearwardly with respect to the proximal housing 1, one or more sloped portions 108 on the slider 100 and/or blocking member 102 cause the slider 100 to move the blocking member 102 radially outwardly, radially past the adjacent portion of the trigger button 10, preferably by camming, to allow the trigger button 10 to be moved forward to the firing position. The slider preferably includes a bump 110 extending radially outwardly which interacts with an inwardly extending foot 112 of the blocking member 102 to retain the slider 100 and the blocking member 102 in the respective positions to enable firing of the injector when the foot 112 is positioned forward of the bump 110 resting against the outside of the slider 100.

The trigger button 10 can now be depressed in a forward direction past the blocking member 102, compressing the trigger spring 11. In the prefiring position, the trigger button 10 retains balls 8 received in locking recess 114 of ram extension 35, interposed with housing latch 15 to prevent firing motion of the ram 7. When the trigger button 10 is moved forward, the balls 8 are pushed out from the locking recess 114 into trigger recess 116, which is preferably a circumferential groove, releasing the ram extension 35 and ram 7, which are driven forward by the compressed spring 6, causing the plunger 45 to eject the insulin from the chamber 50.

In moving of the trigger button 10 to the firing position, a forward-facing portion of the trigger button 10 preferably contacts and moves the slider 100 forward from the release position to the safety position. When the trigger button is released by the user, spring 11 biases and moves the trigger button 10 back to the prefiring position, and the blocking member 102 is allowed to resiliently returned to the blocking position, and the safety mechanism is thus automatically reactivated. In the preferred embodiment, the slider 100 is moved in a first direction, such as distally, from the release position to the safety position, and the trigger button 10 is moved substantially in the first direction towards the firing position to activate the energy source.

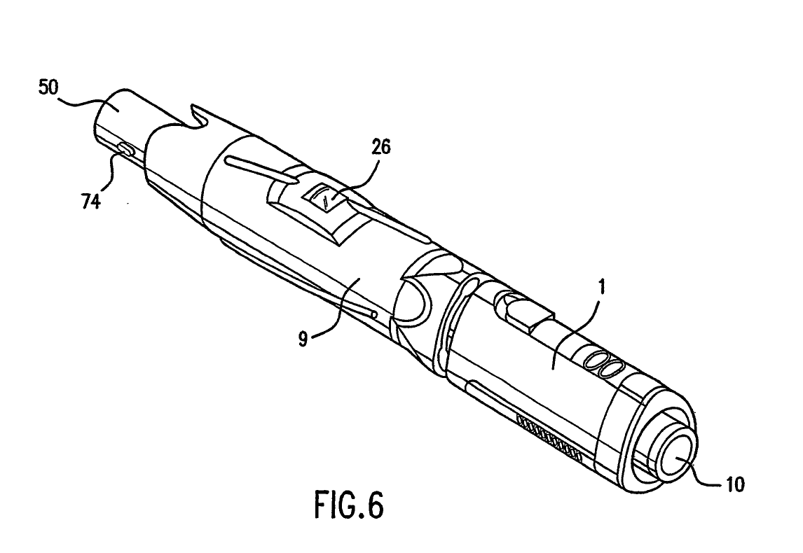

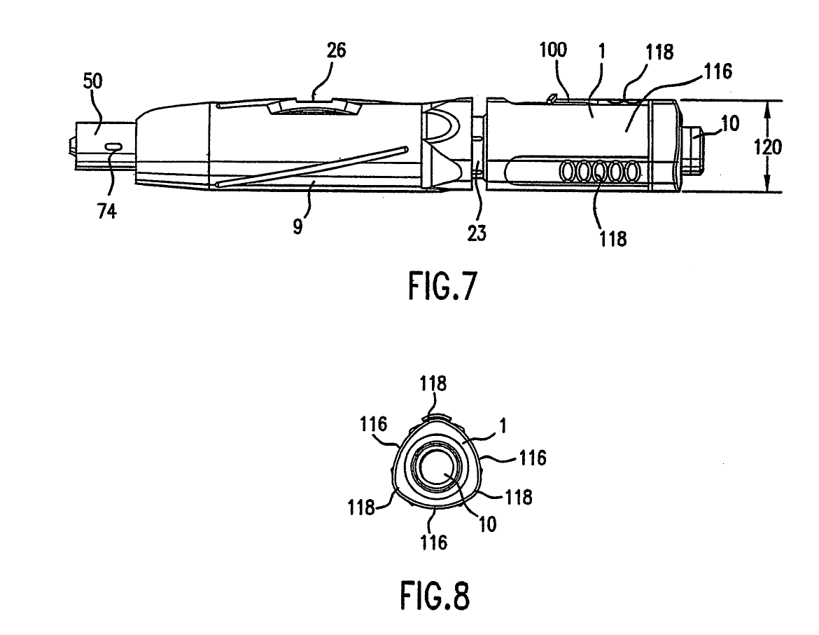

Referring to FIGS. 6-8 the rear housing 1 preferably has an axial cross-section that is generally triangular for facilitating the patients grip during operation of the injector. The cross-section is preferably rounded, with convex sides 116, to comfortably hold in the patient's hand. A lobe 118 protrudes at each apex of the triangular cross-section. The lobes are also preferably rounded and dimensioned for fitting adjacent the inside of the patient's knuckles during the injection and operation of the injector. Preferably, an elastomer or member surface is disposed at the lobes 118 to improve the user's grip. In other embodiments, the elastomeric surface can be disposed over substantially all of the surface that is locate to come into contact with the user's hand during the injection or over substantially the entire rear housing 1. The height 120 of the cross-section from a lobe 118 to an opposite side 116 is preferably about between 0.75 in. and 1.5 in., and more preferably around 1 in. The axial length of the injector is preferably about between 5 in. and 10 in.

In general, the preferred injectors, including the Antares Pharma Vision and similar injectors, administer medication as a fine, high velocity jet delivered under sufficient pressure to enable the jet to pass through the skin. Because the skin is a tissue composed of several layers and the injector is applied to the external surface of the outermost layer, the delivery pressure must be high enough to penetrate all layers of the skin. The layers of skin include the epidermis, the outermost layer of skin, the dermis, and the subcutaneous region. The required delivery pressure is typically about 2500 psi to 3500 psi.

### EXAMPLES

Preferred embodiments of the invention are now illustrated by way of the following examples.

### Example 1

Fifteen type 1 diabetic subjects were included in a study of insulin injection using a Antares Pharma Vision jet injection device. The subjects were eight females and seven males with the following profile: mean age of 30±6 years, mean diabetes duration of 10±5 years, mean body mass index (BMI) of 24.3±2.2 Kg/m², as well as mean blood pressure (BP) of 125±4 mm Hg systolic and 75±5 mm Hg diastolic. Each of the individuals also had been intensively treated since diabetes diagnosis, and the subjects had a mean daily insulin dose of 33±6 U.I. Informed consent was obtained from each subject for continuous subcutaneous glucose monitoring using the Minimed Continuous Glucose Monitoring System (CGMS).

The duration of the study of the subjects was three days. During the first day, each subject used a Novopen Demi-pen device to inject regular human insulin 30 minutes before breakfast, lunch, and dinner. During the second day, each subject used the Antares Pharma Vision jet injection device to inject regular insulin. Finally, on the third day, each subject again used the pen device to inject regular insulin.

During the study, the insulin/carbohydrates ratio was 1/15 CHO, and the mean content of the diet was 430±30 Kcal at breakfast, 860±55 Kcal at lunch, and 660±45 Kcal at dinner, all composed of 56% CHO, 19% proteins, 25% fats.

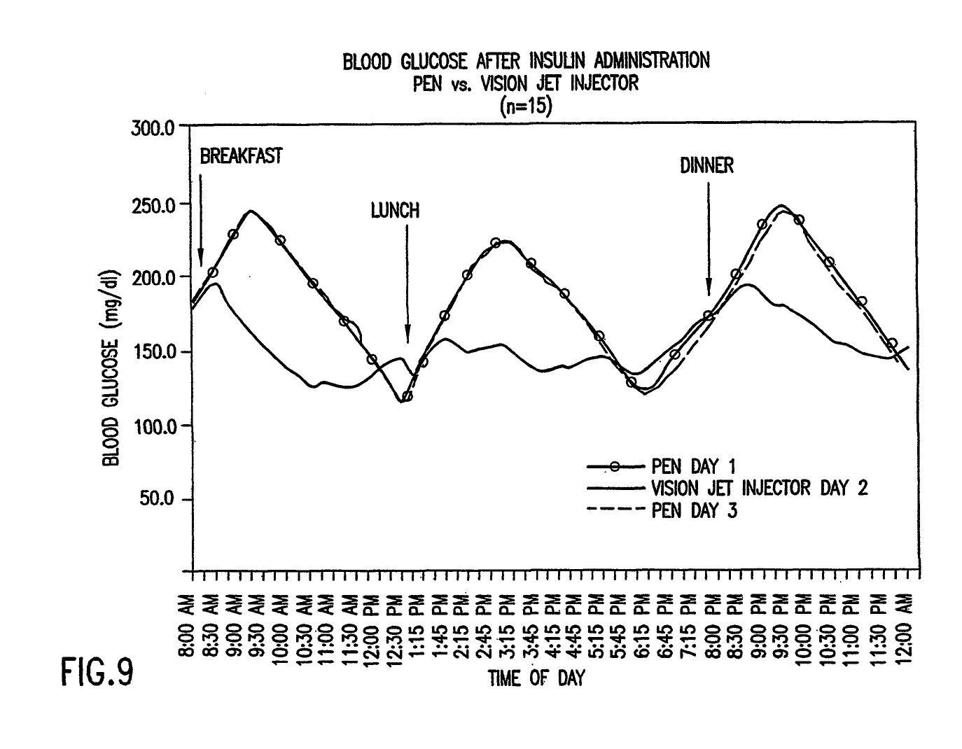

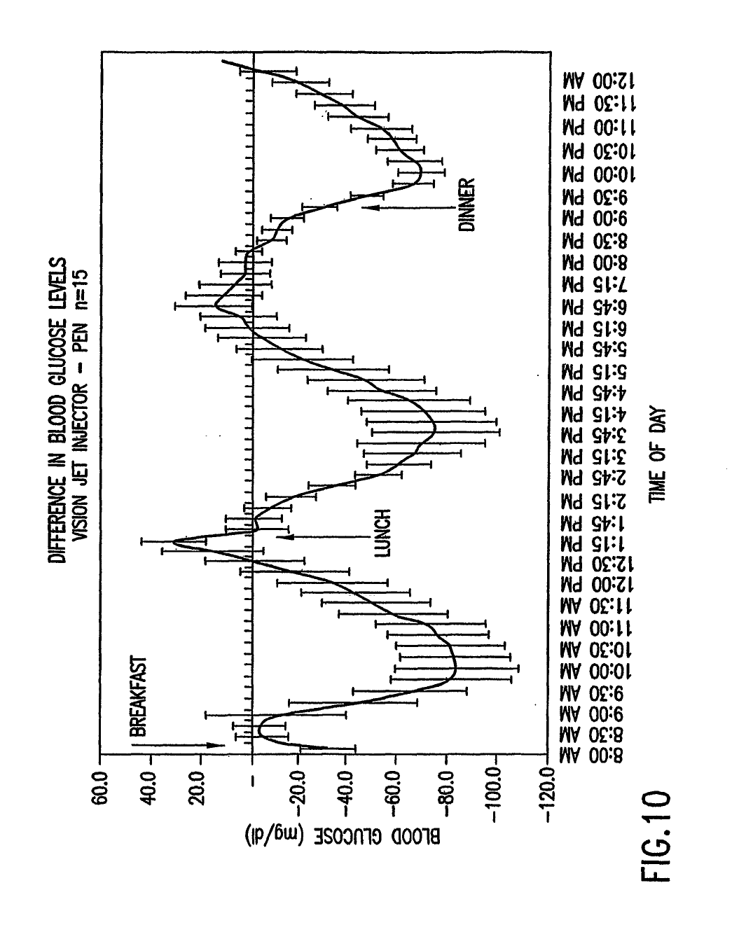

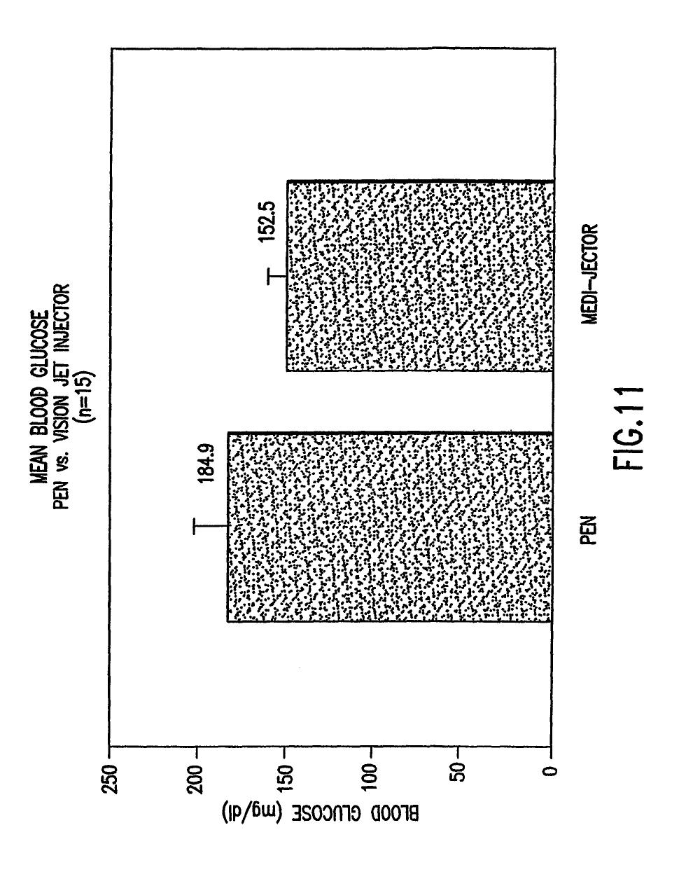

As shown in FIGS. 9-11, the results of the study show that insulin administered by the jet injection device, in comparison to the pen device, produced a significantly lower (p<0.01) glucose profile from 45 to 255 minutes after breakfast-time injection, 45 to 270 minutes after lunchtime injection, and 45 to 240 minutes after dinner-time injection. The maximum blood glucose difference was at 105 minutes after breakfast and dinner, and at 150 minutes after lunch. A significant reduction (p<0.01) in area under the blood glucose curve can also be seen, without lesions in the injection site (abdominal wall) and without a loss in blood glucose control at the end of the dosing period.

Furthermore, a comparison of the blood glucose profile after administration of insulin with the pen device and the Antares Pharma Vision jet injection device demonstrates that the Antares Pharma Vision device produces quicker absorption of regular insulin compared to the absorption profile using the pen device, and concomitantly a significantly lower blood glucose profile without an increase in hypoglycemia after food ingestion.

Accordingly, compared to insulin administration with a needle, the Vision jet injection device demonstrated that the blood glucose profile produced by jet injection of insulin was sustained for one year and that HbA I c levels declined throughout the year of using jet injection. Subjects with reasonable glycemic control as evidenced by HbA1c (≦8.0%) were able to achieve meaningful improvement after changing mode of insulin administration to jet injection. Thus, a needle-free jet injection administration of insulin can be advantageous in reducing the risk of diabetes complications.

### Example 2

This example was conducted to determine whether the improvement in glycemic profile observed in short-term studies of needle-free insulin administration, such as those of Example 1, could be sustained long term, resulting in improvement of HbA1c levels. To document HbA1c levels in subjects using the jet-injector and to measure their blood glucose profile after one year, the following materials and methods were used. Five type 1 diabetic patients (3 females, 2 males) had the following profile: age 34±4 years, diabetes duration 9.5±4.5 years, BMI 23±1.2 Kg/m2, systolic BP 126±6 and diastolic BP 76±3 mmHg, daily insulin dose 36±4 IU/day (70% Regular, 30% NPH). All subjects consented to periodic HbA1c evaluations and 72-hours continuous subcutaneous glucose monitoring.

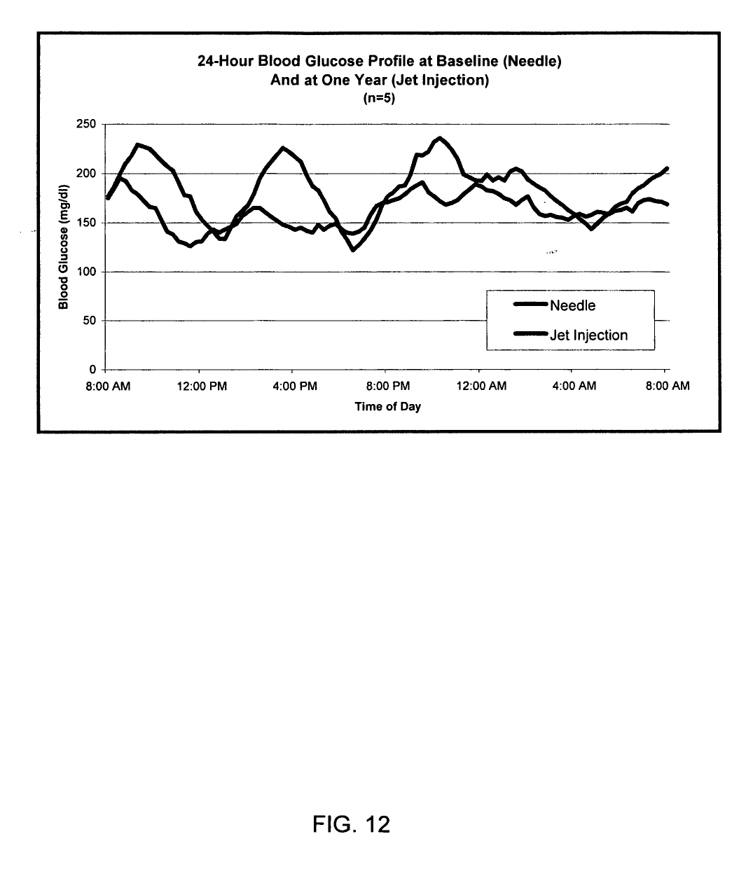

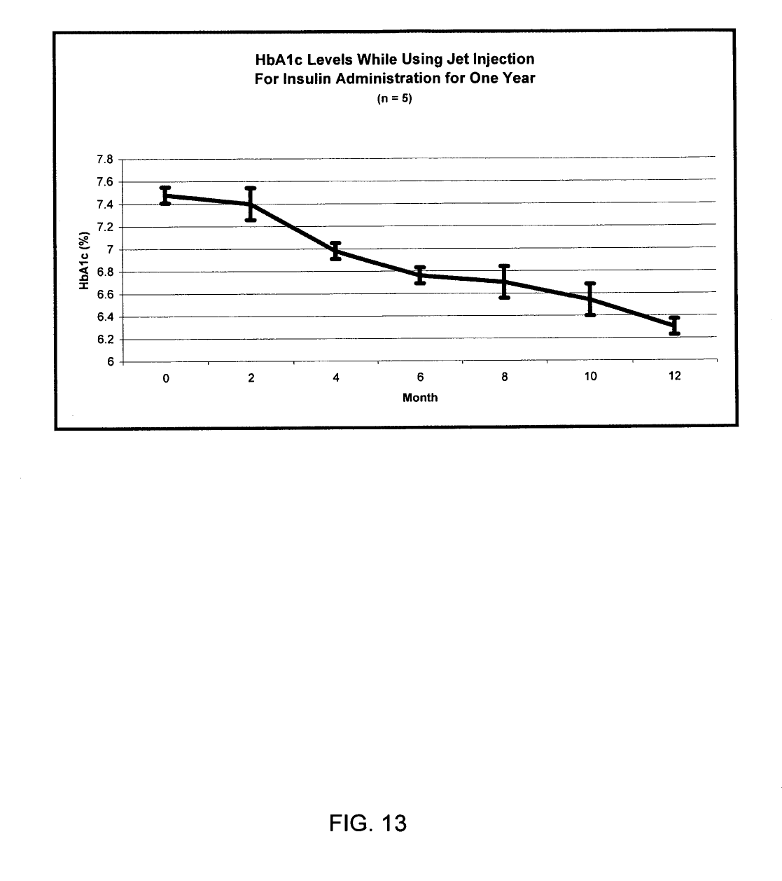

A baseline glucose profile was obtained while subjects used the Novopen Demi-pen needle device. Subjects were switched to a jet-injector for one year, and a blood glucose profile was then obtained at one year. The monitoring periods were performed during working days, with the consumption of a stable diet (breakfast 430±30, lunch 860±55, dinner 660±45 Kcal) with 56% carbohydrates, 19% proteins, 25% fats) and minimal physical activity. Regular-insulin was injected 30 minutes before food consumption, and NPH was injected at bedtime. Results: HbA1c levels decreased from 7.3±0.4% at baseline to 6.7±0.4% after six months and 6.3±0.2% after one year (See FIG. 13). This is a reduction of over 8% after 6 months and about 14% after one year. Daily glucose profiles observed at the end of one year of jet-injection consistently showed lower postprandial blood glucose compared to the baseline (see FIG. 12).

Conclusion: Subjects experienced a continuous decline in HbAlc over one year of jet-injection insulin therapy. Improvements in the blood glucose profile using a jet-injector could be demonstrated with continuous monitoring.

### Example 3

The management of nocturnal NPH insulin is commonly a problem for type 1 diabetic patients because of hypoglycemia risk. The use of a jet injector reduces nocturnal glucose levels and thus reduces the hypoglycemia risk. To compare nocturnal blood glucose after NPH insulin administered alternatively with a pen device (Novopen Demi-pen needle device) and a needle-free jet-injector (Antares Pharma Vision® injector device), the following Materials and Methods were used.

15 type 1 diabetic subjects (7 males, 8 females), age 31±4 and diabetes duration 9±4 years, BMI 23.5±1.8 Kg/m2, systolic BP 130±4 and diastolic BP 78±4 mmHg, were intensively treated since diabetes onset (43±5 I.U. insulin—NPH typically 30% of the total). The mean HbA1c values were 7.0±0.4%. These subjects consented to 72-hour continuous subcutaneous glucose monitoring (Minimed® CGMS device) and to use the pen device the first and the third night and the Vision jet injector the second night of the study. All subjects received NPH in the upper arm at 11:00 pm each night. All the patients otherwise maintained consistent activity, insulin dose and diet during the study.

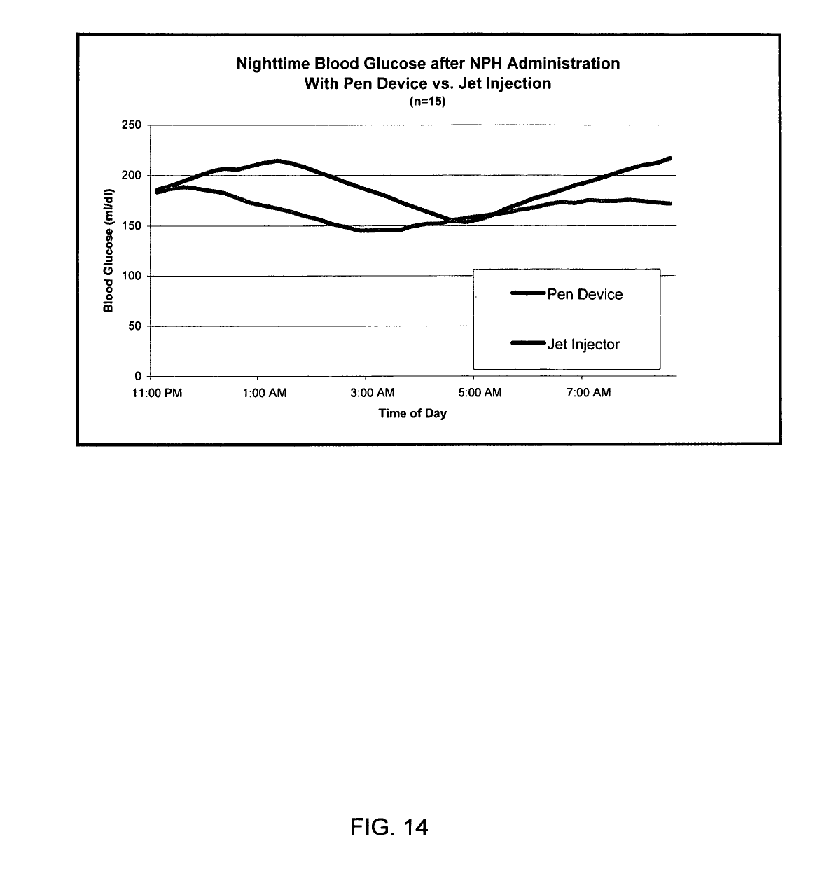

Results: Blood glucose after using the jet injector was significantly lower than that with a pen device between 12:45 am to 3:15 am and between 5:30 am and 8:30 am (p<0.01) (see FIG. 14). Thus, blood glucose reductions were maintained for a period of about 5 to about 8 hours while the patient was sleeping and otherwise inactive. The pen device produced lower but not statistically different blood glucose levels between 4:00 am and 5:00 am. No hypoglycemic episodes were recorded during the study.

Conclusions: The nighttime blood glucose profile was improved using the jet-injection compared to a pen device. Blood glucose control with jet injection was superior at the end of the dosing period, and the blood glucose nadir was less pronounced after jet-injection. Specifically, compared to insulin administration with a needle, the Antares Pharma Vision jet injection demonstrated lower average blood glucose level through the night and a less-pronounced blood glucose nadir in the early morning hours. Thus, the use of the Vision device resulted in a superior blood glucose profile compared to that obtained by using a pen needle. Most notably, the risk of nighttime hypoglycemia can be reduced when using the Vision device. Also, needle-free administration of NPH insulin was well tolerated by all subjects.

While it is apparent that the illustrative embodiments of the invention herein disclosed fulfill the objectives stated above, it will be appreciated that numerous modifications and other embodiments may be devised by those skilled in the art. Therefore, it will be understood that the appended claims are intended to cover all such modifications and embodiments which come within the spirit and scope of the present invention.

What is claimed is:

1. A method for reducing mean blood glucose levels in an insulin dependent patient that is receiving insulin through needle injection, the method comprising administering the insulin to the patient by jet injection rather than by the needle injection or substituting a jet injector for a needle injection assembly for administration of the insulin so that HbA1c levels are reduced by at least 5% over a period of 6 months.

2. The method of claim 1, wherein the jet injector administers about 0.02 mL to 0.5 mL of insulin to the patient within at most about 0.5 seconds and the so that HbA1c levels are reduced by at least 10% over a period of one year.

3. The method of claim 1, wherein the insulin is administered to the patient from a jet injector that comprises:
a jet nozzle configured for firing the insulin in a fluid jet configured and with sufficient velocity to penetrate tissue of the patient to an injection site;
an insulin chamber associated with the nozzle for containing the insulin and feeding the insulin to the nozzle for injection;
a firing mechanism comprising an energy source associated with the insulin chamber for forcing the insulin through the nozzle at said velocity; and
a trigger movable by a user and associated with the firing mechanism for activating the energy source for the forcing of the insulin through the nozzle upon movement of the trigger by the user to a firing position.

4. A method for reducing nocturnal mean blood glucose levels in an insulin dependent patient, which comprises administering insulin to the patient by jet injection prior to bedtime to reduce mean blood glucose through the night and to produce a less-pronounced blood glucose nadir in the early morning hours, thus reducing the risk of nighttime hypoglycemia

5. The method of claim 4 wherein the difference between high and low blood glucose levels during the night is about 25% of the high level or less.

6. The method of claim 5 wherein the high blood glucose level is less than about 200 mg/dL.

7. The method of claim 4 wherein the mean blood glucose levels do not exceed the level at the time of injection for at least 5 hours.

8. The method of claim 4, wherein the insulin is administered to the patient from a jet injector that comprises:
a jet nozzle configured for firing the insulin in a fluid jet configured and with sufficient velocity to penetrate tissue of the patient to an injection site;
an insulin chamber associated with the nozzle for containing the insulin and feeding the insulin to the nozzle for injection;
a firing mechanism comprising an energy source associated with the insulin chamber for forcing the insulin through the nozzle at said velocity; and
a trigger movable by a user and associated with the firing mechanism for activating the energy source for the forcing of the insulin through the nozzle upon movement of the trigger by the user to a firing position.
